(19) United States
(12) Patent Application Publication
(10) Pub. No.: US 2021/0364512 A1
(43) Pub. Date: Nov. 25, 2021

(54) METHOD AND APPARATUS TO PROVIDE CONNECTED, IN-SITU, COMPREHENSIVE, AND ACCURATE LATERAL FLOW ASSAYS

(71) Applicant: Sameh Sarhan, Santa Clara, CA (US)

(72) Inventor: Sameh Sarhan, Santa Clara, CA (US)

(73) Assignee: Xtrava Inc, Santa Clara, CA (US)

(21) Appl. No.: 17/323,780

(22) Filed: May 18, 2021

Related U.S. Application Data
(60) Provisional application No. 63/027,323, filed on May 19, 2020.

(51) Int. Cl. G01N 33/543; B01L 3/00; G01N 35/00; G01N 21/65

(52) U.S. Cl. CPC G01N 33/54386; G01N 33/54373; B01L 3/5023; G01N 35/00029; G01N 21/65; B01L 2200/025; H04L 67/10; B01L 2300/0663; B01L 2300/126; B01L 2300/023; G01N 2035/00108; G01N 2035/00306; B01L 2200/04

(57) ABSTRACT

A method, apparatus and system that includes an environmentally controlled accurate and sensitive general purpose lateral flow assay instrument that can be used throughout the world, in homes, and make-shift emergency centers, including while connected to the internet to receive reference Transmission Raman Spectroscopy signature data, and to transmit test results.

### CROSS-REFERENCE TO RELATED APPLICATION

Priority is claimed under 37 CFR 1.78 and 35 USC 119(e) to U.S. Provisional Application 63/027,323 (XT2005191), filed 19 May 2020, which is incorporated by reference.

### TECHNICAL FIELD

This disclosure relates generally to instruments that determine the presence and/or quantity of chemical compounds. More specifically, this disclosure pertains to automated assay instruments, such as immunoassay instruments, that use the vertical paper based or lateral flow techniques.

### BACKGROUND

For many years, the paper-based assay method, including the lateral flow method has been known for its ability to provide handy determination of the presence and concentration of various substances. Although the most familiar applications for the lateral flow assay method are pregnancy tests, the method is used for a significant variety of additional tests and in many industries, especially the medical industry. For medical purposes, paper-based, including lateral flow assay devices, also accept samples in many forms, such as blood, urine, semen, and saliva. Many diseases and illnesses, such as various types of influenza, can be detected, such as by detecting antibodies created by the patient; so that requisite treatments can begin or be continued with increased confidence.

The principal values of the lateral flow assay method are low cost, portability, and ease of use, including by those who are unskilled in the practice analytical chemistry. Lateral flow assay test strip assemblies can be used in residences, clinical settings, and other locations all over the world, including those in undeveloped countries.

A liquid sample, that may or may not contain a target chemical compound, “analyte” is applied close to one end of a test strip consisting of absorbent material that draws the sample by capillary action through the entire length thereof, wetting the strip as it proceeds.

If and only if the sample contains the target analyte compound, a chemical means keyed to it at a fixed “test line” position along the strip causes a visible “stain” line to be formed, while the remainder of the sample passes to the far end, assisting with capillary action.

Many test strip models also include a different chemical at a “control line” position downstream of the one designed to detect the analyte. This second chemical means reacts to all compounds by forming its own stain line, thus indicating to the user that sufficient sample quantity was introduced.

Notwithstanding the immense value of paper-based and assay methods, there are serious limitations.

The accuracy and sensitivity are far below that obtainable by clinical laboratories. Consequently, detection of the medical condition can be delayed until progress of the illness allows fewer treatment options and/or increased risk of complications or death. Related to the above limitation is the difficulty of identifying a stain line when it is in the process of emerging from the test strip. If a stain line is identified by the user, the analyte is considered as present within the liquid sample. However, owing to low concentration and/or other factors, the stain line can be indistinct or invisible, causing determination to be difficult and/or unreliable, especially for the non-expert user. Even worse than discarding a test, it may be interpreted incorrectly. Owing to manufacturing tolerances of the test strip, which may require a very low cost, the test stain line may be slightly spread instead of being confined to a distinct location. Therefore, professional laboratories, assuming they even exist in underdeveloped regions, sometimes use high-priced image analysis equipment or reflected light instrumentation to capture very light and/or indistinct test stain lines.

There is no portable lateral flow equipment available to automatically send data, such as using the internet, after each test to medical organizations, or governmental organizations to assist with epidemic statistics.

Methods to conveniently re-configure a lateral flow device to test for a newly discovered medical condition or an existing medical condition that has become an epidemic do not exist. Also not in existence is portable lateral flow assay equipment that reports results via the internet, including in real time, that allows authorities to map an epidemic or pandemic.

The instant disclosure describes methods and embodiments to overcome the present lateral flow assay limitations stated above.

The methods and equipment described herein achieve the superior analysis capabilities of expensive optical analysis equipment that could eliminate the need for visual inspection, detect analyte concentration far below that of visual inspection and camera image analysis, and be so inexpensive that they are readily available for all medical offices and nearly all consumers for home use. It is understood that detection of analyte at lower concentrations can permit knowledge of pregnancy, influenza, or other medical conditions at earlier times, when there are additional advantageous treatment options. The positive impact on society of such widespread capability should not be underestimated. Witness the revolution in diabetes care that resulted from accurate home testing of blood glucose levels.

### BRIEF SUMMARY

This Brief Summary is provided as a general introduction to the Disclosure provided by the Detailed Description and Figures, summarizing some aspects of the disclosed invention. It is not a detailed overview of the instant disclosure and should not be interpreted as necessarily identifying key elements of the invention, or otherwise characterizing the scope of the invention disclosed in this Patent Document.

The instant disclosure describes an environmentally controlled, accurate, and sensitive general purpose lateral flow assay instrument that can be used throughout the world, in homes, and make-shift emergency centers, including while wirelessly connected to the internet to receive setup data and transmit test results.

Contained in or associated with the general purpose lateral flow assay instrument (hereinafter termed “Health Diagnostic Device”), are the following features that eliminate or mitigate the serious limitations of existing equipment and systems listed in the Background section of the instant disclosure:

Increased sensitivity and accuracy are obtained by: 1) use of an electronic reader contained within the instrument that projects electromagnetic energy of selected wavelengths and intensity on one surface of the test strip and measures intensity and spectral content emanating from the opposite surface using broadband detectors; 2) provides the test strip with optimum temperature and humidity just prior to insertion of the sample; 3) analyzes the electromagnetic energy that has passed through the test strip using built-in broadband detectors in addition to spectroscopic subsystems including Transmission Raman Spectroscopy (“TRS”) aided by reference Raman spectroscopy signatures received prior to tests; 4) uses machine learning deep-learning algorithms to continuously refine accuracy and/or sensitivity; and 5) can make use of a special reference stain line that registers the characteristics of minimum relevant sample antigen content.

The ability to communicate data to medical centers permits medical personnel to have instantaneous alert of conditions that are dangerous to the patient and/or the community. In times of epidemic or pandemic, such data can be automatically shared with appropriate governmental agencies to assist with decision making on resources and civil rules. The Health Diagnostic Device contains all the necessary storage, data handling, and communications electronics to send test data through the internet and other services, normally with a smart-phone as intermediary.

In addition to transmitting test results, the Health Diagnostic Device reads RFID data and/or barcode data contained within the test strip cassette pertaining to the type of test and other pertinent information. This information may include setup information such as reference data, optimum testing temperature and testing humidity. Additionally, the Health Diagnostic Device may send the RFID identification data to the test strip vendor or medical group in order to receive up-to-date settings information. Reviewing the long-existing Lateral Flow Assay method, a liquid sample, that may or may not contain a target chemical compound, “analyte” is applied close to one end of a test strip consisting of absorbent material that draws the sample by capillary action through its entire length thereof, wetting the strip as it proceeds. There are two major positions along the strip. At the first position is a pre-deposited first compound that combines with the analyte compound if present. This first compound is carried with the sample fluid to the second major position whether or not the keyed analyte is present, i.e. whether or not its composition is changed. The second major position, called the test line position, is pre-deposited with a second chemical compound that is fixed at this position. If the first compound is in an unchanged composition state, it is not attracted to the second compound and is carried to the far end of the strip by the sample fluid. If the first compound is in a changed composition state, it is attracted to the second compound, and collects at the test line position, causing a reaction of the second compound that creates a colored stain line that is visible upon inspection as a positive test for presence of the analyte.

Instead of requiring visual inspection or camera image analysis, which is useless when the analyte concentration is small enough that accumulation of staining compound is only inside the test strip, the Health Diagnostic Device includes methods disclosed in Provisional Patent Application No. 62/942,694 filed 2 Dec. 2019—to measure the extinction amount of light passing through the test strip at the test line region, without regard to the location of stain line compound—outside or inside of the test strip.

By projecting light toward one side of the strip, measuring how much light emerges on the opposite side, and comparing extinction at the test line region with that at adjacent regions, very small staining compound concentrations are detected. Moreover, by choosing a wavelength that is absorbed by the staining compound and varying the intensity of the incident light, the total amount of this compound is measured, not just the area of a shadow. The result is a wide dynamic range quantitative measurement, with an ability to distinguish small changes whether there is a large amount of staining compound or minute amount. Also, by providing color and intensity that varies depending upon illumination of the test line region and adjacent regions, further increased sensitivity can be achieved.

Moreover, as a means to gather additional information, electronic sensors and instrumentation are capable of dynamic measurements during the wicking process instead of a single measurement when wicking is complete.

An important feature of the Health Diagnostic Device described in the instant disclosure is the inclusion of Raman Spectroscopy as well as common spectroscopy to detect the presence of target analyte or analytes in the test line region.

As for common spectroscopy, every substance produces its own spectral pattern of emission and/or absorption lines when sufficient electromagnetic energy is introduced at the frequencies of these lines. Common spectroscopy has been used routinely to detect and quantify substances in the laboratory and in distant stars for about a century. These spectral patterns can occur at frequencies ranging from far infrared through ultraviolet, depending upon the substance. Therefore, the common spectrographic components used in the Health Diagnostic Device are designed to be frequency agile over a very broad spectrum and combined with broadband light sources. Such common spectrographic configurations supplement the use of multi-colored light sources with broad band detectors.

The Raman scattering method identifies the presence of substances using a narrow spectral band technique that detects the spectrum of vibrational frequencies instead of emission frequencies resulting from orbital transitions. Each substance has its own signature of vibration frequencies.

Raman spectroscopy is implemented using a narrow band light source of arbitrary frequency, such as a laser or narrow-band light emitting diode. When the laser light passes within the substance, most of what occurs is elastic scattering, called Rayleigh scattering. As no energy is exchanged, light having the light source frequency is emitted in all directions. A small percentage of the scattering, however, is inelastic owing to the vibration occurring within the substances. The scattered light from these collisions will be slightly higher or lower than the light source frequency by amounts related to quantized variation of the sample molecule's ground state electronic energy levels induced by its vibration.

When the Rayleigh-scattered light is notch-filtered and remaining Raman-scattered light fed through a narrow band spectroscope, the vibration spectral signature shows which substances are present within the sample.

In order to identify a disease at the earliest possible time, the instrument must detect the smallest possible amounts of target analyte, which may be an antibody produced by the test subject directed to the disease. Therefore, the instrument must receive as high an intensity of the Raman scattered light as possible to produce a useful Raman spectral signature. Normally, only a very small amount of Raman-scattered light is produced, and the scattering is in all directions; so only a small percentage of that amount reaches the spectroscope.

Various methods are used to maximize the amount of Raman-scattered light and to direct this light to the narrow band spectroscope. One method that greatly increases the production of Raman scattering is called Surface Enhanced Raman Scattering (“SERS”). During lateral flow and vertical paper-based immunoassays, the analyte is first caused to coat (be adsorbed to) nanoparticles (termed Raman Tags) that are specially designed for this purpose. The enhancement factor can be as much as 100 billion, which means this technique may detect single molecules of the analyte.

Another technique is to use the Transmission Raman method and reflect as much scattered light as possible toward the narrow band spectroscope using a unidirectional mirror. The Transmission Raman method is used to detect all substances in the sample, not just the ones that are near the surface.

Using the TRS method, whether or not SERS is also employed, the Health Diagnostic Device described in the instant disclosure needs no moving parts. The laser can irradiate a wide enough width of the lateral flow strip to account for all test line position manufacturing tolerances. It can even irradiate several test lines at once, where the Raman spectral signature will show the presence of all analytes at once.

Finally, the Health Diagnostic Device will increase sensitivity by operating in a differential mode by comparing, using deep machine learning methods, its locally obtained Raman Signature with that of a well-equipped laboratory derived from a minimal sample analyte quantity, received prior to the test.

Other aspects, features and advantages of the invention will be apparent to those skilled in the art from the following Disclosure.

### BRIEF DESCRIPTION OF DRAWINGS

For a more complete understanding of this disclosure and its features, reference is now made to the following description, taken in conjunction with the accompanying figures, in which:

FIG. 1 illustrates an example prior art Lateral Flow Assay test strip system 100 used to determine the presence or absence of a chosen substance, also known as the analyte, within a liquid.

FIG. 2 illustrates an example method to employ the same lateral flow assay test method but register the result without need for manual or instrumented optical inspection.

FIG. 3 shows a test strip cassette suitable for insertion into the environmental chamber within an automated electronic reader, here contained within a sealed bag and storage/shipping box.

FIG. 4 shows additional detail of the test strip and cassette, and how it is applied.

FIG. 5 shows the test strip cassette inserted into a section of the environmental chamber.

FIG. 6 is identical to FIG. 5 except for showing an auxiliary sample collection chamber that fits over the sample application region.

FIG. 7 also shows the test strip cassette inserted into the environmental chamber, in this view showing the environmental generation and measurement components.

FIG. 8 shows the environmental chamber, with cassette inserted, residing within the Health Diagnostic Device base module/enclosure.

FIG. 9 shows the Health Diagnostic Device base module/enclosure from each end.

FIG. 10, an extension of FIG. 8, shows the Health Diagnostic Device configured to include a Transmission Raman Spectrograph.

FIG. 11 shows a typical Raman spectroscopy signature.

FIG. 12 is a flow chart that shows operation of the subject Health Diagnostic Device.

### DETAILED DESCRIPTION

The various figures, and the various embodiments used to describe the principles of the present invention in this patent document are by way of illustration only and should not be construed in any way to limit the scope of the invention. Those skilled in the art will understand that the principles of the invention may be implemented in any type of suitably arranged device or system.

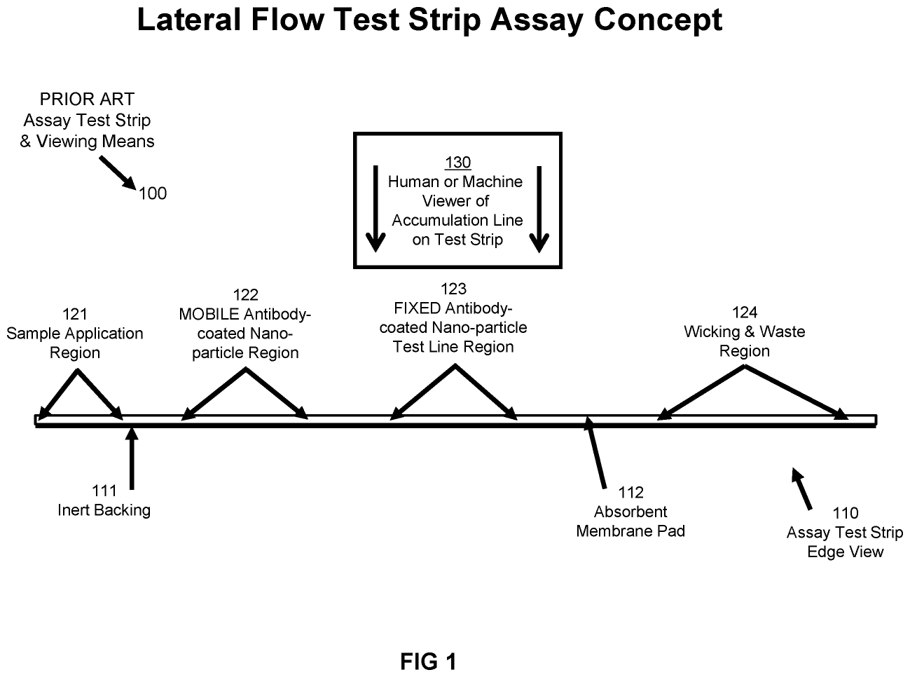

FIG. 1 illustrates an example prior art Lateral Flow Assay test strip system 100 used to determine the presence or absence of a chosen substance, also known as the analyte, within a liquid. Said test strip consists of a chemically inert backing 111 that may also provide physical strength and stability, and an attached absorbent membrane 112, whose function is to transport liquids from Sample Application Region 121 to Wicking and Waste Region 124, after passing through Regions 122 and 123 described below.

Located in Region 122 is a deposition of non-reactive metallic or non-metallic nanoparticles that are coated with an antibody substance that is specifically chosen to conjugate with the analyte. These nanoparticles are not permanently attached to the absorbent membrane and will therefore be carried by the fluid sample as it travels along the membrane. Clustered near the center of Region 123 is a deposition of identical coated nanoparticles, but in this case, they are permanently attached to the absorbent membrane and will not travel with the sample fluid. The region to the right of Region 123, especially Region 124, is used to as an extension of the strip to help draw the sample fluid through Region 123 via capillary action and to provide a storage location for the sample fluid and waste products.

If the sample fluid applied at Region 121 does not include the Analyte, when it reaches Region 122, the antibody substance is unchanged as the nanoparticles travel with the sample fluid to Region 123. When they arrive at Region 123, they do not react with or attach to the nanoparticles there and continue to travel with the sample fluid to Region 124.

If the sample fluid applied at Region 121 contains the Analyte that corresponds with the antibody substance coating the nanoparticles present at Regions 122 and 123, there is a different scenario. When the sample fluid reaches Region 122, it carries the nanoparticles toward Region 123 as in the previous case, but at Region 122 and during the travel to Region 123, the Analyte, according to its concentration, reacts (“conjugates”) with the antibody substance on some fraction of the nanoparticles.

When the nanoparticles reach Region 123, remaining conjugated but unattached coated nanoparticles attach themselves to the immobile unconjugated coated nanoparticle there. These nanoparticles are therefore trapped in Region 123 and accumulate, creating a visible line that indicates presence of the Subject Analyte in the sample fluid. This visible line can be viewed by Human or machine/instrumented viewing means 130.

Test strip 110 is normally contained within an enclosure (not shown), having a suitable opening at sample application region 121 to apply a sample. There is also a transparent region of the enclosure, sometimes referred to as a results window, at test line region 130. Viewer 130 is shown on the side of test strip 110 not having backing 111, but said backing could be transparent, and the results window and viewer 130 could instead be on the backing side.

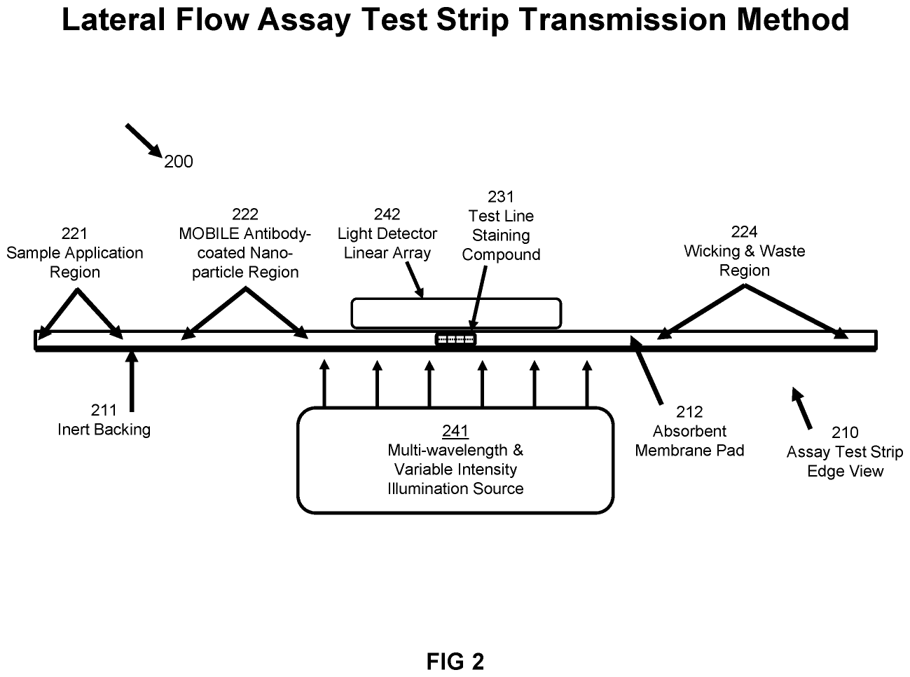

FIG. 2, the approach disclosed in Application No. 62/942,694, illustrates an example method to employ the same lateral flow assay test method but register the result without need for manual or instrumented optical inspection. Moreover, quantitative measurement of the analyte is further facilitated.

The human, incident light reflected or camera-based image analyzer is replaced with an illumination source 241 on one side of test strip 210 and light detector 242 on the opposite side. Illumination source 241 and light detector 242 could be separate equipment units or contained within an integrated test fixture (not shown). Inert backing 211 could be transparent. Illumination source 241 illuminates enough of test strip 210 to cover the region where the staining compound can accumulate 231, plus the adjacent regions for comparison. Illumination source 241 could provide light having a multiplicity of wavelengths and a multiplicity of intensities. Wavelength and intensity could vary with time, position along the test strip, or both.

Light detector 242 could consist of a linear array of many individually addressable optical detector “pixels”. These detector pixels are normally broadband but could also have narrow band spectral response. The detectors in array 242 could be very close to the test strip surface, permitting intensities to be measured without need for optical focusing components. By reading separate light values at each position along the test strip, an extinction curve can be generated to be used to derive analyte sample presence and concentration.

By projecting light toward one side of test strip 210, measuring how much light emerges on the opposite side, and comparing extinction at the test line region with that at other regions, very small staining compound concentrations are detected. Moreover, by choosing a wavelength that is absorbed by the staining compound and varying the intensity of the incident light, the total amount of this compound is measured, not just the area of a shadow. Additionally, by providing wavelength and intensity that varies depending upon illumination of the test line region and adjacent regions, additional sensitivity can be achieved.

Moreover, through use of variable intensity and wavelength lighting, this method is applicable to a wide variety of test strip analyte chemical processing and test stain line development processes, all using a single instrument model.

While the preceding paragraphs and FIG. 2 describe a multi-wavelength illumination source coupled through a lateral flow test strip to a broadband light detector, it may be advantageous under some circumstances to employ a broadband illumination source and selective wavelength detectors, also known as spectrometers. In such case, block 241 could be labeled “Broadband Variable Intensity Illumination Source”, and block 242 could be labeled “Spectrometer Array”.

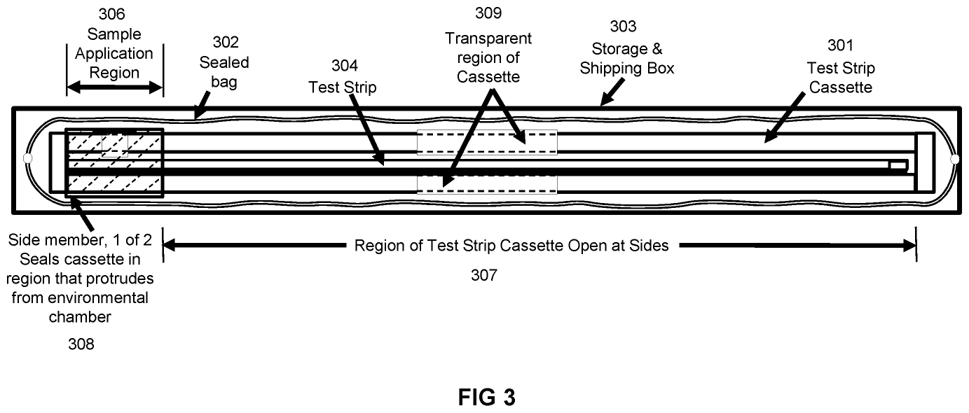

FIG. 3 shows a test strip cassette 301 suitable for insertion into the environment chamber (not shown) of a Health Diagnostic Device. Until ready for use, it is kept in a sealed bag 302 within a storage and shipping box 303. The figure shows the edge view of the test strip 304. One major distinguishing characteristic between the subject cassette and prior art is that the subject cassette is designed to be inserted into an environmental chamber, with only the Sample Application Region 306 protruding.

Another major distinguishing characteristic is that this cassette encloses the test strip 304 only at its top and bottom; the sides are open 307 except for the portion that remains on the outside of the environmental chamber. The open sides of this cassette allow the test strip to achieve equilibrium of the temperature and humidity chosen for the environmental chamber. The closed sides 308 in the sample application region maintain the environmental seal of the chamber.

The cassette walls are transparent 309 in the region where the stain lines appear shortly after the sample is inserted, allowing transmission of light and visibility on both faces of the test strip.

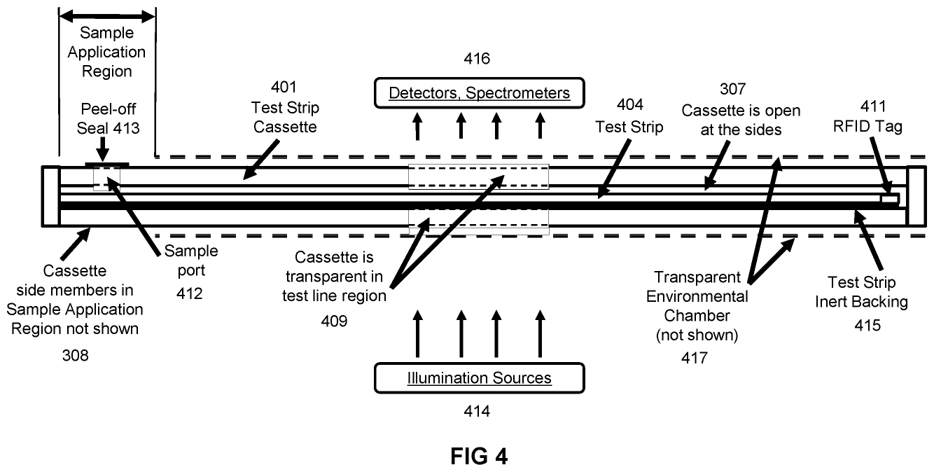

FIG. 4 shows additional detail of the test strip 404 and cassette 401 and how it is applied. The test strip may 404 include an RFID tag 411, which may include data on which tests are supported, and setup information such as what temperature and humidity are needed, settings of illumination sources 414, detectors, spectrometers 416, and time and place of manufacture. An abundance of RFID-sourced information is critical for situations where access to the internet is not available.

Not shown in the figure is that the test strip may include a bar code in addition to or instead of the RFID tag 411.

The cassette walls may or may not be transparent, but in the stain line region of the test strip, they must be transparent 409 to accommodate illumination from one side and detection of light that passes through.

The sample port 412 includes a peel off seal 413 that should not be removed until immediately before the fluid sample is inserted. Although not shown, the seal could be reinstated right after the sample is inserted in order to help maintain environmental chamber 417 temperature and humidity while the sample is flowing and stain lines are developing.

The illumination sources 414 could supply lighting of various wavelengths and intensities, programmed on a per-test basis. After passing through test strip 404 with its transparent inert backing 415, lighting from sources 414 is input to the detectors, spectrometer 416.

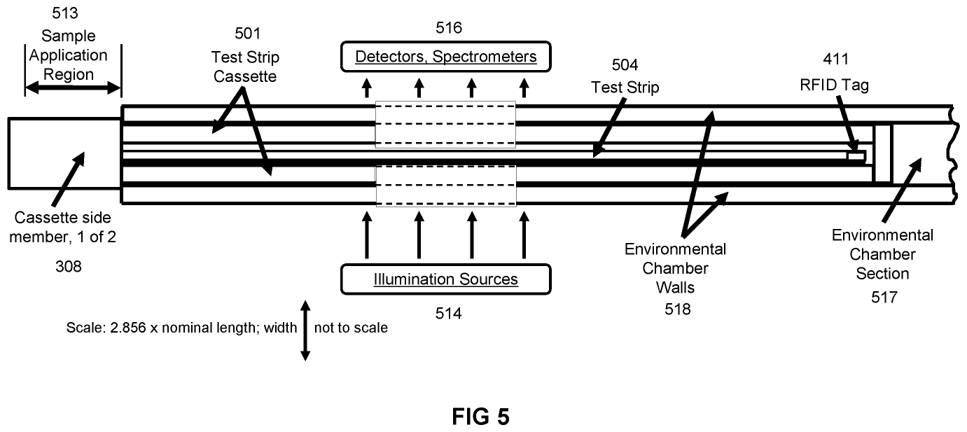

FIG. 5 shows the test strip cassette 501 inserted into a section of the environmental chamber 517. For clarity, only the top and bottom walls 518 are shown; this chamber 517 is completely enclosed except for the left end, open to accommodate entry of the cassette. The environmental chamber walls 518 may or may not be transparent, but in the stain line region of the test strip 504, they must be transparent to accommodate illumination 514 from one side and detection 516 of light that passes through.

Not shown in the figure is that the section of the environmental chamber wall 518 in proximity with the detectors and/or spectrometers 516 may have lensing properties for focusing. The chamber wall near the illumination sources 514 may also include lensing properties.

When the cassette 501 is in place, the section containing the test strip sample application region 513 protrudes outside the chamber 517 and provide a vapor seal. The protruding cassette 501 section is slightly larger in cross section than the chamber opening to prevent it from being inserted too far and to facilitate the vapor seal.

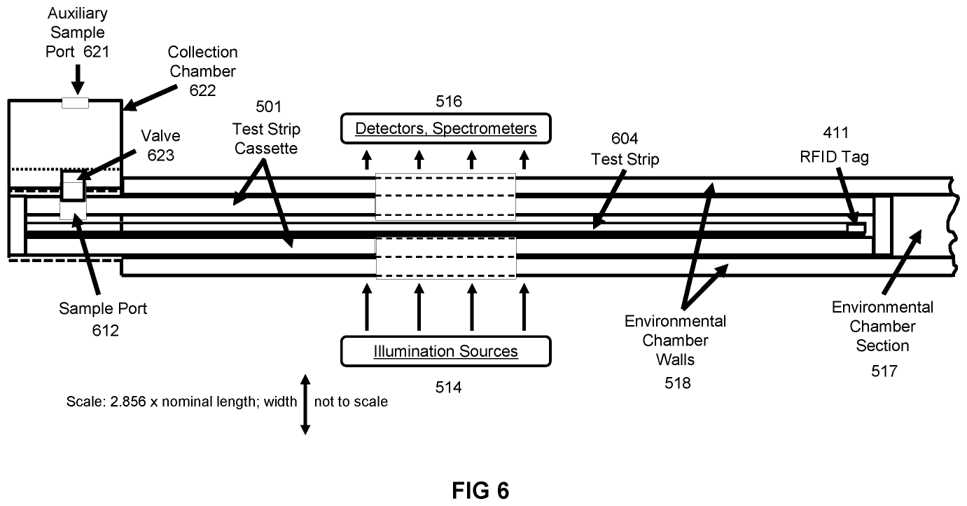

FIG. 6 is identical to FIG. 5 except for showing an auxiliary sample port 621 and collection chamber 622 that fits over the sample port 612. Thus, different types of samples can be applied sequentially using valve 623 to the same test strip 604 (such as serology, saliva and urinalysis. Each type of sample can receive the correct solvent or other chemical to accommodate the test being performed.

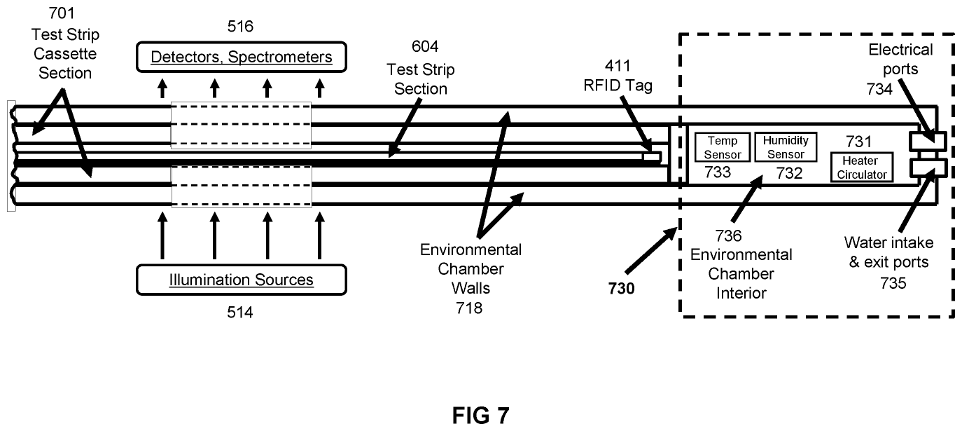

FIG. 7 also shows the test strip cassette 701 inserted into the environmental chamber 718. Here, the sample application region 513 is not shown. Instead, the chamber 718 region 730 containing environmental generation and measurement components are shown. These components include a heater/air circulator 731, a humidity sensor 732, and temperature sensor 733. Not shown is a possible heating wire distributed on the outside of environmental chamber 718. At the end of environmental chamber are also electrical ports 734, and water intake/exit ports 735.

Not obvious in this view is that air within the environmental chamber interior 736 can pass freely to all regions of the test strip.

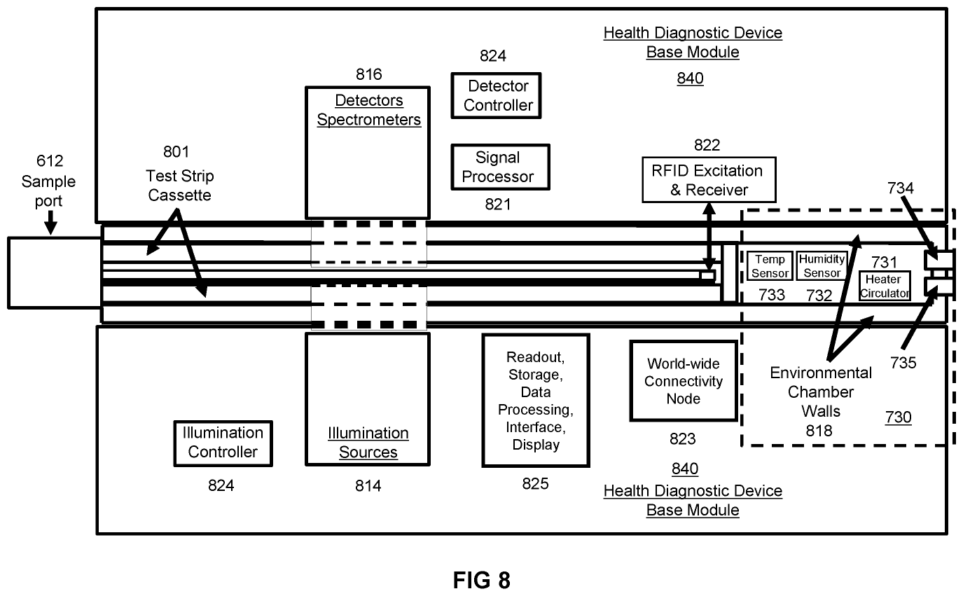

FIG. 8 shows the environmental chamber 818, with cassette 801 inserted, residing within Health Diagnostic Device base module/enclosure 840. The environmental chamber 818 can be removed for cleaning.

Not obvious in this view is that the Health Diagnostic Device base module/enclosure is a single piece, with the environmental chamber fitting into a slot.

The Health Diagnostic Device base module/enclosure 840 contains all components necessary for its function, including illumination 814, measurement 816, detector measurement controller 824, signal processing 841, RFID retrieval 822, RF communication 823, illumination controller 824, and memory storage/data processor/display/battery/power management.

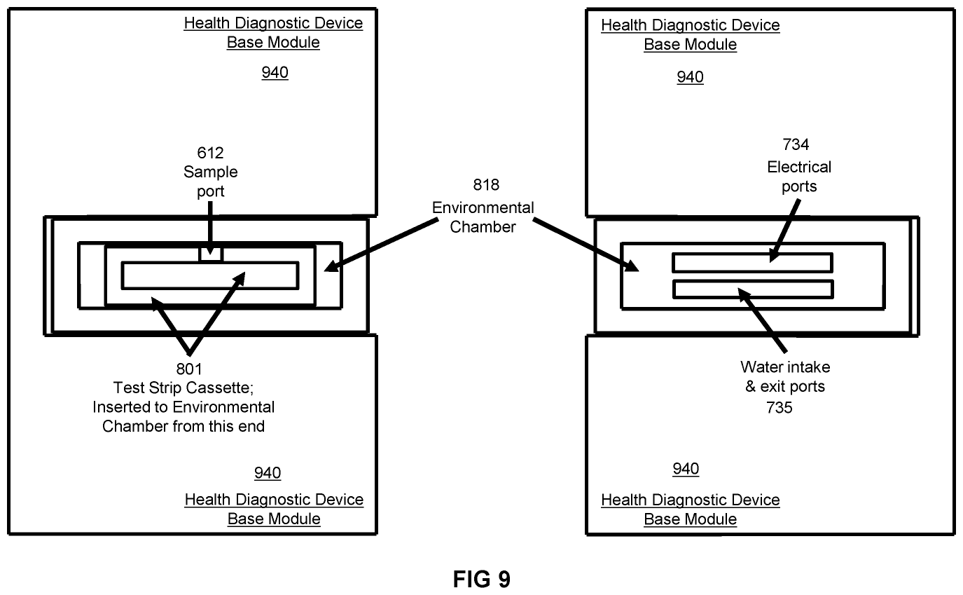

FIG. 9 shows the Health Diagnostic Device base module/enclosure from each end. Finally, it is clear how air can circulate freely among all regions of the environmental chamber 818 and test strip 604. Covering of cassette 801 and chamber 818 opening is not shown in the sample port view.

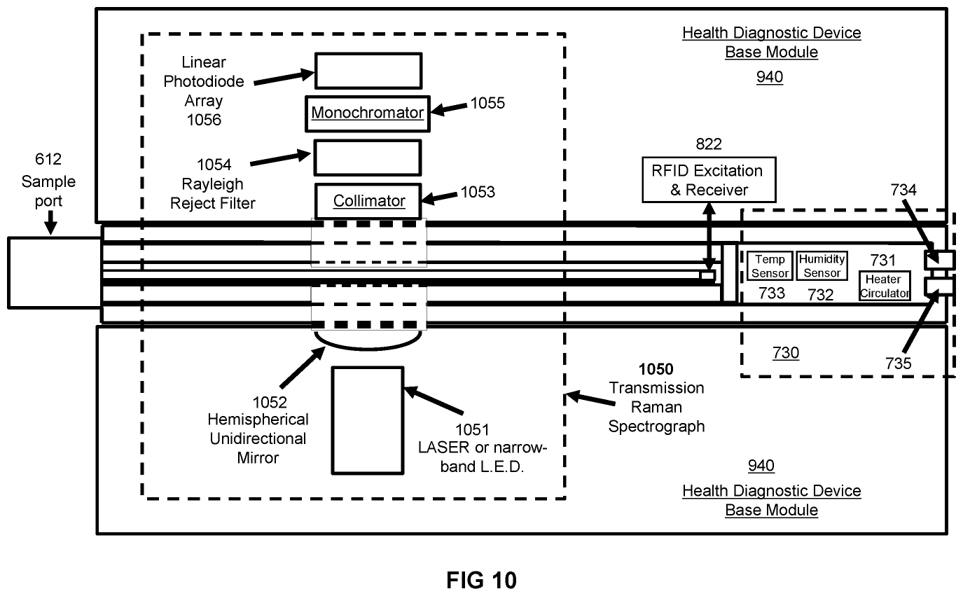

FIG. 10 is an extension of FIG. 8 and is the exemplary embodiment. It shows an example configuration of a Transmission Raman Spectrograph (“TRS”) 1050. Not visible in the figure but determined by the type of nanoparticles present in the test line region of the lateral flow test strip is that this TRS 1050 may be a surface enhanced type of Raman spectrograph.

The laser or narrow-band LED light source 1051 irradiates the sample window portion of the test strip. Prior to passing through the environmental chamber 818, cassette 801, and test strip 604, the sourced light 1051 passes through a hemispherical unidirectional mirror 1052, whose function is to re-direct backscattered light toward the collimator 1053.

Rayleigh reject 1054 filter rejects the strong elastically scattered light that is exactly on the sourced light frequency. The monochromator 1055 is the spectrograph-directs the Raman-scattered light toward the linear photodiode array 1056 in slightly different directions, depending upon frequency.

By polling the output of each diode in the linear diode array 1056, a Raman spectral signature is obtained to compare with the sample signature obtained via the internet or other means from the laboratory equipment.

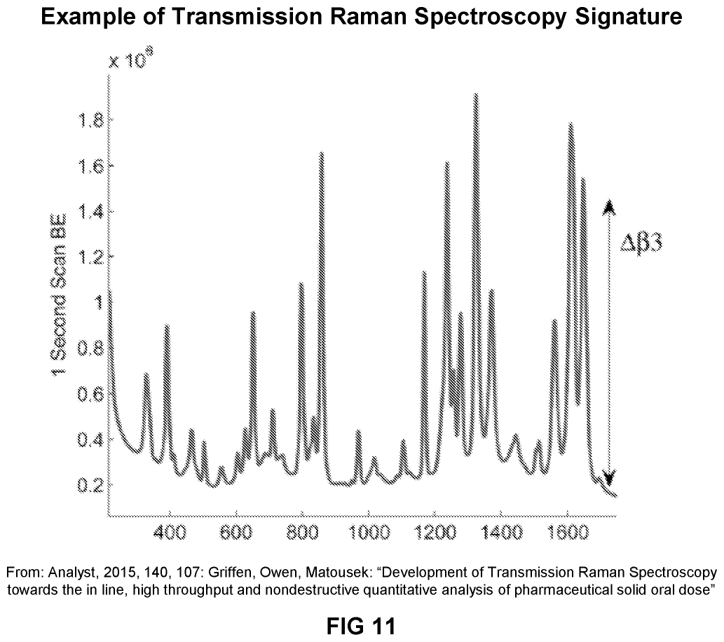

FIG. 11 shows a typical Raman spectroscopy signature. The horizontal axis represents the offset frequency (shown as wave number) from the carrier. If a line is present at the correct frequency offset, the target analyte is interpreted as present.

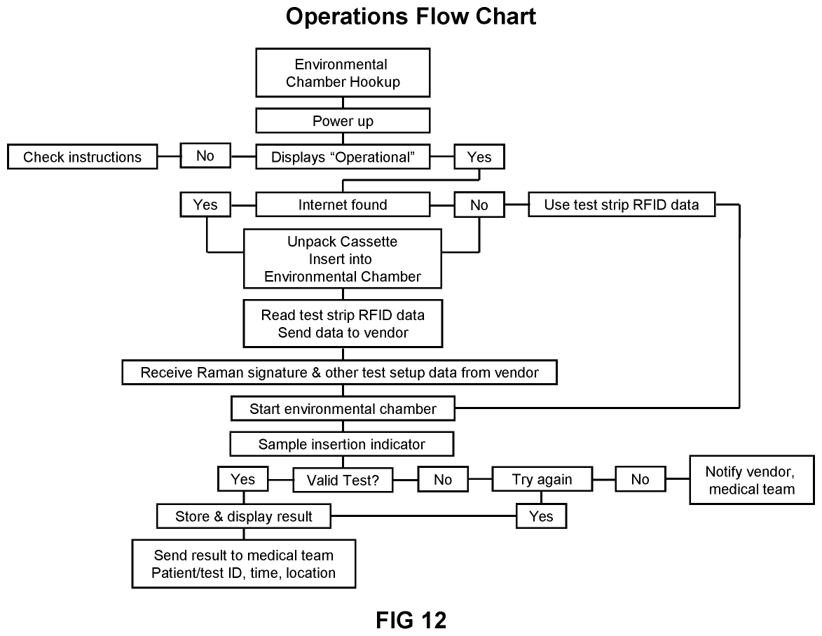

FIG. 12 is a flow chart that shows operation of the subject Health Diagnostic Device.

The details provided in the above description describe particular implementations of the systems for performing the measurements described. Other embodiments could be implemented in any other suitable manner. For example, particular voltages, frequencies, noise levels, gains, resistances, capacitances, and other values may be described. These values are for illustration only. It may be advantageous to set forth definitions of certain words and phrases used throughout this patent document. The term “couple” and its derivatives refer to any direct or indirect communication between two or more elements, whether or not those elements are in physical contact with one another. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, have a relationship to or with, or the like.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. An automated method to determine the presence and concentration of target chemical compounds in a sample introduced to a lateral flow immunoassay test strip consisting of
a cassette that houses the test strip,
an environmental chamber that contains the cassette,
a light source directing light onto one surface of the test strip's stain line region,
a light detector that receives light from the opposite surface of the test strip's stain line region; where
a backing material of the test strip is transparent to light,
the cassette and the environmental chamber are both transparent to light at the stain line regions of the test strip;
and a hardware and a firmware that analyzes a signal output of the light detector; whereby
after the sample is introduced to the test strip, light from the light source passes through the stain line region of the test strip that has been brought to a standardized environment and impinges upon the light detector whose output signal is analyzed by the hardware and firmware to determine the presence and/or concentration of target chemical compounds.

2. The method of claim 1, where the light source intensity and spectral content can be varied.

3. The method of claim 1, where temperature and/or humidity are controlled within the environmental chamber.

4. The method of claim 1, where the cassette is removable from the environmental chamber.

5. The method of claim 1, where there is also a wireless cloud connection.

6. The method of claim 1, where following introduction of the sample, the hardware and firmware analyze the sample multiple times or continuously.

7. The method of claim 1, where the light source and detector consists of a Transmission Raman Spectrograph.

8. The method of claim 1, where said target chemical compounds consist of influenza or corona virus antibodies.

9. The method of claim 5, where the wireless cloud connection is used to receive information to facilitate a test, further analyze the test result, or report test result to patient, medical personnel, and/or governmental health authorities.

10. A portable apparatus to automatically determine the presence and concentration of target chemical compounds in a sample introduced to a lateral flow immunoassay test strip consisting of
a cassette that houses the test strip,
an environmental chamber that contains the cassette,
a light source directing light onto one surface of the test strip's stain line region,
a light detector that receives light from the opposite surface of the test strip's stain line region; where
a backing material of the test strip is transparent to light,
the cassette and the environmental chamber are both transparent to light at the stain line regions of the test strip, and
a hardware and a firmware that analyzes a signal output of the light detector,
a controller for the light source,
a controller for the light detector,
a wireless cloud connectivity node, and
an overall housing that contains all the foregoing components; whereby
after the sample is introduced to the test strip, light from the light source passes through the stain line region of the test strip that has been brought to a standardized environment, impinges upon the light detector whose output signal is analyzed by the hardware and firmware to determine the presence and/or concentration of target chemical compounds, and the resulting data is communicated to the cloud.

11. The apparatus of claim 10, where the light source intensity and spectral content can be varied.

12. The apparatus of claim 10, where temperature and/or humidity are controlled within the environmental chamber.

13. The apparatus of claim 10, where the cassette is removable from the environmental chamber, and the environmental chamber is removable from the overall housing.

14. The apparatus of claim 10, where a collection chamber and valve assembly is attached to a sample input port of the cassette to facilitate the sequential introduction of different types of samples.

15. The apparatus of claim 10, where following introduction of the sample, the hardware and firmware analyze the sample multiple times or continuously.

16. The apparatus of claim 10, where the light source and detector consists of a Transmission Raman Spectrograph.

17. The apparatus of claim 10, where said target chemical compounds consist of influenza or corona virus antibodies.

18. The apparatus of claim 10, where the wireless cloud connection is used to receive information to facilitate a test, further analyze the test result, or report test result to patient, medical personnel, and/or governmental health authorities.
